SEQUENCE LISTING (21 sequences)

SEQ ID NO: 1 - protein, 80 aa, Artificial Sequence
  Synthetic
MPQERQQVTRKHYPNYKVIVLNDDFNTFQHVAACLMKYIPNMTSDRAWELTNQVHYEGQAIVWVGPQEQAELYHEQLLRA

SEQ ID NO: 2 - protein, 21 aa, Artificial Sequence
  Synthetic
GIVEQCCTSICSLYQLENYCN

SEQ ID NO: 3 - protein, 30 aa, Artificial Sequence
  Synthetic
FVNQHLCGSHLVEALYLVCGERGFFYTPKT

SEQ ID NO: 4 - protein, 4 aa, Artificial Sequence
  Synthetic
NYCN

SEQ ID NO: 5 - protein, 13 aa, Artificial Sequence
  Synthetic
FVNQHLCGSHLVE

SEQ ID NO: 6 - protein, 9 aa, Artificial Sequence
  Synthetic
RGFFYTPKT

SEQ ID NO: 7 - protein, 8 aa, Artificial Sequence
  Synthetic
ALYLVCGE

SEQ ID NO: 8 - protein, 4 aa, Artificial Sequence
  Synthetic
GIVE

SEQ ID NO: 9 - protein, 13 aa, Artificial Sequence
  Synthetic
QCCTSICSLYQLE

SEQ ID NO: 10 - protein, 17 aa, Artificial Sequence
  Synthetic
GIVEQCCTSICSLYQLE

SEQ ID NO: 11 - protein, 23 aa, Artificial Sequence
  Synthetic
NLCNIPCSALLSSDITASVNCAK

SEQ ID NO: 12 - protein, 16 aa, Artificial Sequence
  Synthetic
NTDGSTDYGILQINSR

SEQ ID NO: 13 - protein, 15 aa, Artificial Sequence
  Synthetic
IVSDGNGMNAWVAWR

SEQ ID NO: 14 - protein, 12 aa, Artificial Sequence
  Synthetic
FESNFNTQATNR

SEQ ID NO: 15 - protein, 12 aa, Artificial Sequence
  Synthetic
GYSLGNWVCAAK

SEQ ID NO: 16 - protein, 9 aa, Artificial Sequence
  Synthetic
GTDVQAWIR

SEQ ID NO: 17 - protein, 7 aa, Artificial Sequence
  Synthetic
WWCNDGR

SEQ ID NO: 18 - protein, 7 aa, Artificial Sequence
  Synthetic
HGLDNYR

SEQ ID NO: 19 - protein, 8 aa, Artificial Sequence
  Synthetic
CELAAAMK

SEQ ID NO: 20 - protein, 5 aa, Artificial Sequence
  Synthetic
TPGSR

SEQ ID NO: 21 - protein, 129 aa, Artificial Sequence [sequence omitted]

(19) United States
(12) Patent Application Publication
(10) Pub. No.: US 2022/0324910 A1
(43) Pub. Date: Oct. 13, 2022

(54) PROTEIN-WIDE MODIFICATION OF ASPARTATES AND GLUTAMATES

(71) Applicant: Quantum-Si Incorporated, Guilford, CT (US)

(72) Inventors: Omer Ad, Madison, CT (US); Haidong Huang, Madison, CT (US)

(73) Assignee: Quantum-Si Incorporated, Guilford, CT (US)

(21) Appl. No.: 17/709,917

(22) Filed: Mar. 31, 2022

Related U.S. Application Data
(60) Provisional application No. 63/169,374, filed on Apr. 1, 2021.

(51) Int. Cl. C07K 7/06; C07D 401/12; C07K 1/02; C12P 21/02

(52) U.S. Cl. CPC C07K 7/06; C07D 401/12; C07K 1/02; C12P 21/02

(57) ABSTRACT

The present disclosure is related to peptides comprising modified aspartic acid and glutamic acid moieties, methods of making such peptides, and methods of using such modified peptides to selectively direct cleavage of peptide bonds. Selective peptide bond cleavage is advantageous in peptide sequencing applications, such as automated peptide sequencing applications.

### RELATED APPLICATION

This application claims priority under 35 U.S.C. § 119(e) to U.S. Provisional Application, U.S.S.N. 63/169,374, filed Apr. 1, 2021, which is incorporated herein by reference.

### REFERENCE TO A SEQUENCE LISTING SUBMITTED AS A TEXT FILE VIA EFS-WEB

This application contains a Sequence Listing which has been submitted in ASCII format via EFS-Web and is hereby incorporated by reference in its entirety. Said ASCII copy, created on May 27, 2021, is named R070870108US01-SEQ-DFC and is 6,295 bytes in size.

### BACKGROUND

Proteomics has emerged as an important and necessary complement to genomics and transcriptomics in the study of biological systems. The proteomic analysis of an individual organism can provide insights into cellular processes and response patterns, which lead to improved diagnostic and therapeutic strategies. The complexity surrounding protein structure, composition, and modification present challenges in determining large-scale protein sequencing information for a biological sample.

### SUMMARY

In one aspect, provided herein is a peptide comprising one or more instances of Formula

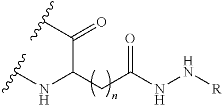

or a salt thereof, wherein: each R is independently aryl, heteroaryl, or —C(O)Rₐ; each Rₐ is independently branched or unbranched, cyclic or acyclic alkyl, branched or unbranched, cyclic or acyclic heteroalkyl, aryl, or heteroaryl; and each n is independently 1 or 2.

In certain embodiments, Formula (I) has the structure of:

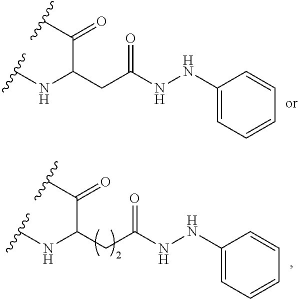

or a salt thereof.

In certain embodiments, Formula (I) has the structure of:

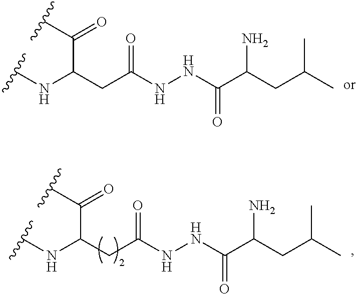

or a salt thereof.

In certain embodiments, Formula (I) has the structure of Formula (II):

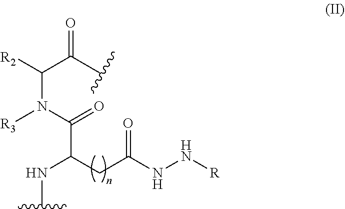

or a salt thereof, wherein: each R2 is independently branched or unbranched, cyclic or acyclic alkyl, branched or unbranched, cyclic or acyclic heteroalkyl, aryl, or heteroaryl;
each R3 is —H, or is combined with R₂ to form a 5-membered heterocyclic ring; and
each n is independently 1 or 2.

In another aspect, provided herein is a method for cleaving a peptide bond (i.e., an amide bond between a nitrogen and a carbonyl), comprising contacting a first peptide comprising a moiety of Formula (I) or Formula (II) with an aminopeptidase enzyme to obtain a second peptide comprising one or more instances of Formula (III):

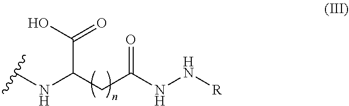

or a salt thereof.

In another aspect, provided herein is a method for modifying an aspartic acid residue or a glutamic acid residue in a peptide, the method comprising coupling a first peptide comprising a moiety of Formula (V):

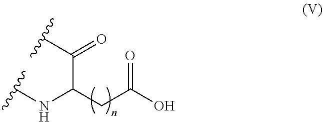

or a salt thereof; wherein n is 1 or 2;
with a compound of Formula (VI):

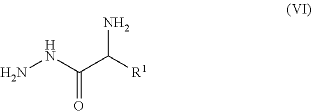

or a salt thereof;
wherein R₁ is cyclic or acyclic alkyl, cyclic or acyclic heteroalkyl, aryl, or heteroaryl;
to obtain a second peptide comprising a moiety of Formula (VII):

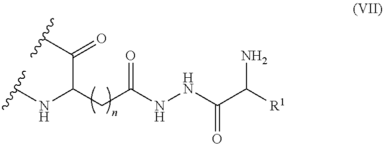

or a salt thereof.

In certain embodiments, the moiety of Formula (VII) has the structure of:

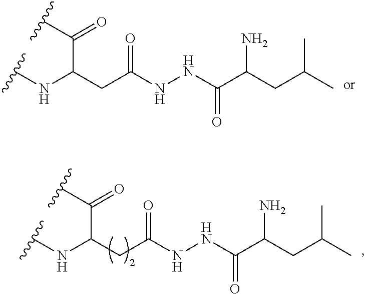

or a salt thereof.

In certain embodiments, the moiety of Formula (V) does not bind to a binder, and the moiety of Formula (VII) does bind to the enzymatic binder. In certain embodiments, the first peptide and the second peptide further comprise an N-terminal amine, and the binder selectively binds to the moiety of Formula (VII) in favor of the N-terminal amine.

### BRIEF DESCRIPTION OF THE DRAWINGS

FIG. 1 shows schemes representing solid-phase vs. solution-phase peptide activation methods.

FIGS. 2A-2B show a scheme and LCMS trace representing the complete labeling of all peptides formed during the gluC digestion of insulin.

FIG. 3 shows the automation compatible protocol used to obtain libraries bearing C-terminally activated Asp/Glu peptides.

FIG. 4 shows a tryptic digest of a capped lysozyme.

FIG. 5 shows a sample preparation using a small protein.

### DETAILED DESCRIPTION

In some aspects, the present disclosure relates to the discovery of compositions and methods useful in peptide sequencing techniques The inventors have recognized and appreciated that differential binding interactions can provide an additional or alternative approach to conventional labeling strategies in peptide sequencing. Conventional peptide sequencing can involve labeling each type of amino acid with a uniquely identifiable label. This process can be laborious and prone to error, as there are at least twenty different types of naturally occurring amino acids in addition to numerous post-translational variations thereof. In some aspects, the present disclosure relates to the discovery of techniques involving the use of amino acid recognition molecules, or “binders”, which differentially associate with different types of amino acids to produce detectable characteristic signatures indicative of an amino acid sequence of a peptide. Accordingly, aspects of the application provide techniques that do not require peptide labeling and/or harsh chemical reagents used in certain conventional peptide sequencing approaches, thereby increasing throughput and/or accuracy of sequence information obtained from a sample.

In particular, the present disclosure is related to peptides comprising modified aspartic acid and glutamic acid moieties, methods of making such peptides, and methods of using such modified peptides to selectively direct cleavage of peptide bonds. Selective peptide bond cleavage is advantageous in peptide sequencing applications, such as automated peptide sequencing applications.

### Modified Peptides

In one aspect, provided herein is a peptide comprising one or more instances of Formula (I):

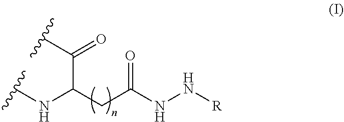

or a salt thereof, wherein:
each R is independently aryl, heteroaryl, or —C(O)Rₐ;
each Rₐ is independently branched or unbranched, cyclic or acyclic alkyl, branched or unbranched, cyclic or acyclic heteroalkyl, aryl, or heteroaryl; and
each n is independently 1 or 2.

In certain embodiments, n is 1. In certain embodiments, n is 2.

In certain embodiments, R is aryl. In certain embodiments, R is phenyl. In certain embodiments, R is heteroaryl. In certain embodiments, R is selected from: benzimidazole, adenine, cytosine, and pyrimidine. In certain embodiments, R is —C(O)Rₐ.

In certain embodiments, R comprises polyethyleneglycol (PEG).

In certain embodiments, Rₐ is branched or unbranched, cyclic or acyclic alkyl. In a particular embodiment, Rₐ is branched alkyl. In another particular embodiment, Rₐ is unbranched alkyl. In another particular embodiment, Rₐ is cycloalkyl.

In certain embodiments, Rₐ is branched or unbranched, cyclic or acyclic heteroalkyl. In a particular embodiment, Rₐ is branched heteroalkyl. In another particular embodiment, Rₐ is unbranched heteroalkyl. In another particular embodiment, Rₐ is heterocycloalkyl (i.e., heterocycyl).

In certain embodiments, Rₐ is aryl. In certain embodiments, Rₐ is heteroaryl.

In certain embodiments, R has the structure:

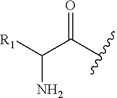

or a salt thereof, wherein each R₁ is independently branched or unbranched, cyclic or acyclic alkyl, branched or unbranched, cyclic or acyclic heteroalkyl, aryl, or heteroaryl;

In certain embodiments, R₁ is independently branched or unbranched, cyclic or acyclic alkyl. In a particular embodiment, R₁ is branched alkyl. In another particular embodiment, R₁ is unbranched alkyl. In another particular embodiment, R₁ is cycloalkyl.

In certain embodiments, R₁ is branched or unbranched, cyclic or acyclic heteroalkyl. In a particular embodiment, R₁ is branched heteroalkyl. In another particular embodiment, R₁ is unbranched heteroalkyl. In another particular embodiment, R₁ is heterocycloalkyl (i.e., heterocycyl).

In certain embodiments, R₁ is a natural amino acid side chain (e.g., a sidechain of glycine, alanine, valine, leucine, isoleucine, methione, phenylalanine, tryptophan, serine, threonine, glutamine, tyrosine, cysteine, lysine, arginine, histidine, aspartic acid, or glutamic acid). In a particular embodiment, R₁ is isobutyl.

In certain embodiments, R₁ is aryl. In certain embodiments, R₁ is heteroaryl.

In certain embodiments, R₁ comprises polyethyleneglycol (PEG).

In certain embodiments, Formula (I) has the structure of:

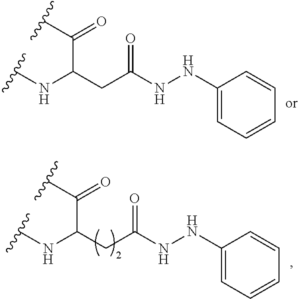

or a salt thereof.

In certain embodiments, Formula (I) has the structure of:

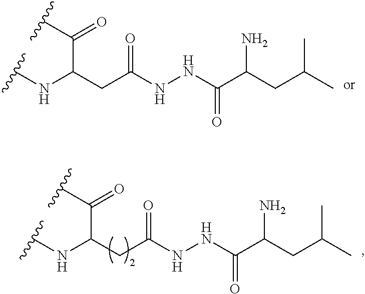

or a salt thereof.

In certain embodiments, Formula (I) has the structure of Formula (II):

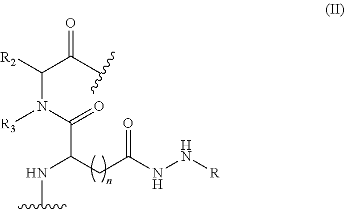

or a salt thereof, wherein:
each R₂ is independently branched or unbranched, cyclic or acyclic alkyl, branched or unbranched, cyclic or acyclic heteroalkyl, aryl, or heteroaryl;
each R₃ is —H, or is combined with R₂ to form a 5-membered heterocyclic ring; and
each n is independently 1 or 2.

In certain embodiments, n is 1. In certain embodiments, n is 2.

In certain embodiments, R is defined according to embodiments of Formula (I).

In certain embodiments, R₂ is independently branched or unbranched, cyclic or acyclic alkyl. In a particular embodiment, R₂ is branched alkyl. In another particular embodiment, R₂ is unbranched alkyl. In another particular embodiment, R₂ is cycloalkyl.

In certain embodiments, R₂ is branched or unbranched, cyclic or acyclic heteroalkyl. In a particular embodiment, R₂ is branched heteroalkyl. In another particular embodiment, R₂ is unbranched heteroalkyl. In another particular embodiment, R₂ is heterocycloalkyl (i.e., heterocycyl).

In certain embodiments, R₂ is a natural amino acid side chain (e.g., a sidechain of glycine, alanine, valine, leucine, isoleucine, methione, phenylalanine, tryptophan, serine, threonine, glutamine, tyrosine, cysteine, lysine, arginine, histidine, aspartic acid, or glutamic acid).

In certain embodiments, R₂ is aryl. In certain embodiments, R₂ is heteroaryl.

In certain embodiments, R₃ is -H. In certain embodiments, R₃ is combined with R₂ to form a 5-membered heterocyclic ring (e.g., a pyrrolidine ring).

### Methods of Cleavage

In another aspect, provided herein is a method for cleaving a peptide bond (i.e., an amide bond between a nitrogen and a carbonyl), comprising contacting a first peptide comprising one or more moieties of Formula (II) with an aminopeptidase enzyme to obtain a second peptide comprising one or more instances of Formula (III):

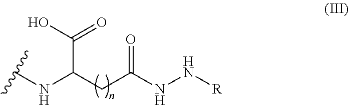

or a salt thereof. In particular, the moieties of Formula (II) are converted to moieties of Formula (III) by cleavage of a peptide bond.

In certain embodiments, the aminopeptidase enzyme is hTET, Vpr, or pfuTET.

In certain embodiments, the method comprises contacting the peptide comprising Formula (II) with the aminopeptidase enzyme for about 1-180 minutes, e.g., about 1-120 minutes, about 1-60 minutes, about 5-60 minutes, about 5-45 minutes, about 10-45 minutes, or about 15-30 minutes.

In certain embodiments, the percent yield of the second peptide is in the range of about 10-100%, about 20-90%, about 50-90%, or about 50-75%.

In certain embodiments, the peptides comprising moieties off Formula (II) and Formula (III) further comprise protected amino acid residues. Such protected amino acid residues include protected cysteine residues. In certain embodiments, the peptides comprising moieties of Formula (II) and Formula (III) further comprise one or more moieties of Formula (IV):

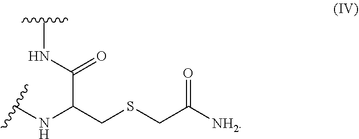

In certain embodiments, the peptides described herein are conjugated to one or more additional molecule, wherein such additional molecules are useful for immobilizing the peptide on a surface, or for facilitating immobilization of the peptide. In certain embodiments, the peptides described herein are conjugated to DNA. Such conjugation products may comprise chemical moieties such as amides, esters, alkyl or heteroalkyl chains, and molecules such as biotin and streptavidin.

### Methods of Manufacture

In another aspect, provided herein is a method for modifying an aspartic acid residue or a glutamic acid residue in a peptide, the method comprising coupling a first peptide comprising a moiety of Formula (V):

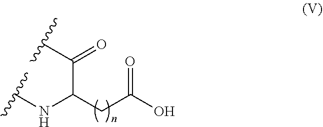

or a salt thereof, wherein n is 1 or 2;
with a compound of Formula (VI):

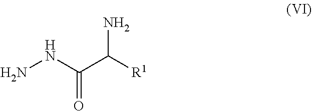

or a salt thereof;
wherein R₁ is cyclic or acyclic alkyl, cyclic or acyclic heteroalkyl, aryl, or heteroaryl; to obtain a second peptide comprising a moiety of Formula (VII):

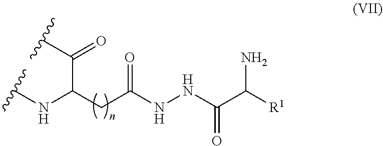

or a salt thereof.

In certain embodiments, n is 1. In certain embodiments, n is 2.

In certain embodiments, R₁ is independently branched or unbranched, cyclic or acyclic alkyl. In a particular embodiment, R₁ is branched alkyl. In another particular embodiment, R₁ is unbranched alkyl. In another particular embodiment, R₁ is cycloalkyl.

In certain embodiments, R₁ is branched or unbranched, cyclic or acyclic heteroalkyl. In a particular embodiment, R₁ is branched heteroalkyl. In another particular embodiment, R₁ is unbranched heteroalkyl. In another particular embodiment, R₁ is heterocycloalkyl (i.e., heterocycyl).

In certain embodiments, R₁ is a natural amino acid side chain (e.g., a sidechain of glycine, alanine, valine, leucine, isoleucine, methione, phenylalanine, tryptophan, serine, threonine, glutamine, tyrosine, cysteine, lysine, arginine, histidine, aspartic acid, or glutamic acid). In a particular embodiment, R₁ is isobutyl.

In certain embodiments, R₁ is aryl. In certain embodiments, R₁ is heteroaryl.

In certain embodiments, R₁ comprises polyethyleneglycol (PEG).

In certain embodiments, the moiety of Formula (VII) has the structure of:

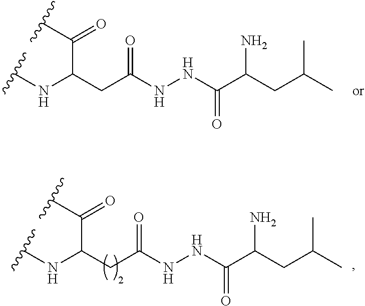

or a salt thereof.

Moieties of Formula (VII) are useful for binding to specific binders, and the structure of R₁ can be tuned to increase binding selectivity with specific binders. The binders are capable of cleaving, facilitating cleavage of, or directing cleavage of, a proximal peptide bond, e.g., cleaving a peptide comprising a moiety of Formula (II) into a peptide comprising a moiety of Formula (III) as shown above.

In certain embodiments, the moiety of Formula (V) does not bind to a binder, and the moiety of Formula (VII) does bind to the binder. In certain embodiments, the binder binds more strongly to the moiety of Formula (VII) binds than to the moiety of Formula (V).

In certain embodiments, the first peptide and the second peptide further comprise an N-terminal amine, and the binder selectively binds to the moiety of Formula (VII) in favor of the N-terminal amine.

In certain embodiments, the binder is teClpS, which as the sequence:

### MPQERQQVTRKHYPNYKVIVLNDDFNTFQHVAACLMKYIPNMTSDRAWELTNQVHYE GQAIVWVGPQEQAELYHEQLLRA (SEQ ID NO: 1).

In some embodiments, the selectivity and/or the efficiency of the cleavage are correlated with the size of the first peptide. In certain embodiments, peptides having a molecular weight greater than about 60 Da are cleaved more selectively and/or efficiently. For example, peptides having a molecular weight of greater than about 60 Da do no undergo the same degree of peptide backbone cleavage under cleavage conditions as compared to peptides having a molecular weight of less than about 60 Da. In certain embodiments, the first peptide has a molecular weight in the range of 60-80 Da, or 70-90 Da, or 80-100 Da, or 100-200 Da, or 150-250 Da, or 200-400 Da, or 300-500 Da, or 400-600 Da, or 500-700 Da, or 600-800 Da, or 700-900 Da, or 800-1000 Da. In a particular embodiment, the first peptide has a molecular weight in the range of 60-500 Da.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas

Sorrell, Organic Chemistry, University Science Books, Sausalito, 1999;Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

When a range of values (“range”) is listed, it encompasses each value and sub-range within the range. A range is inclusive of the values at the two ends of the range unless otherwise provided. For example “C₁-6 alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₇ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₈ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F , —CH₂CHF₂, —CH₂CF₃, or benzyl (Bn)).

The term “cycloalkyl” refers to a monocyclic or bicyclic saturated or partially unsaturated (non-aromatic) cyclic alkyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the ring system, as valency permits.

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and for 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The terms “heterocyclyl”, “heterocyclic” and “heterocycloalkyl” refer to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1,2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 p electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C6 aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C14 aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

The term “heteroaryl” refers to a radical of a 5-14 membered monocyclic or polycyclic (e.g., bicyclic, tricyclic) 4n+2 aromatic ring system (e.g., having 6, 10, or 14 p-electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1,2,3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1,2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not limited in any manner by the exemplary substituents described herein.

Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb}R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO_{w}R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃ —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
   wherein:
each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀alkenyl, heterC₁₋₂₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₃, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ perhaloalkyl, C₁₋₂₀ alkenyl, C₁₋₂₀ alkynyl, heteroC₁₋₂₀alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{cc})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₃, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀alkyl, heteroC₁₋₁₀alkenyl, heteroC₁₋₁₀alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents are joined to form ═O or ═S; wherein X⁻ is a counterion;
each instance of R^{ee} is, independently, selected from C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₁₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH,—SC₁₋₆ alkyl, —SS(C₁₋₆ alkyl), —C(═O)(C₁₋₆ alkyl), —CO₂, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═ONH₂, —C(═NH)O, —OC(═NH)(C₁₋₆ alkyl), —OC(═NH)OC₁₋₆ alky, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N((C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃ —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl; or two geminal R^{gg} substituents can be joined to form ═O or ═S; and
each X⁻ is a counterion.

In certain embodiments, each carbon atom substituent is independently halogen, substituted (e.g., substituted with one or more halogen) or unsubstituted C₁₋₆ alkyl,—OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, each carbon atom substituent is independently halogen, substituted (e.g., substituted with one or more halogen) or unsubstituted C₁₋₁₀ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, each carbon atom substituent is independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

In certain embodiments, the molecular weight of a carbon atom substituent is lower than 250, lower than 200, lower than 150, lower than 100, or lower than 50 g/mol. In certain embodiments, a carbon atom substituent consists of carbon, hydrogen, fluorine, chlorine, bromine, iodine, oxygen, sulfur, nitrogen, and/or silicon atoms. In certain embodiments, a carbon atom substituent consists of carbon, hydrogen, fluorine, chlorine, bromine, iodine, oxygen, sulfur, and/or nitrogen atoms. In certain embodiments, a carbon atom substituent consists of carbon, hydrogen, fluorine, chlorine, bromine, and/or iodine atoms. In certain embodiments, a carbon atom substituent consists of carbon, hydrogen, fluorine, and/or chlorine atoms. The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from -0R^{aa}, —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

The term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═S)OR^{aa}, —SC(═S) N(R^{bb})₂, —SC(═O)SR^{aa}—SC(═O)OR^{aa}, —SC(═O)N(R^{bb})₂, and —SC(═O)R^{aa}, wherein R^{aa} and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{bb}CO₂R^{aa}, —NR^{bb}C(═O)N(R^{bb})₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein. The term “sulfonyl” refers to a group selected from —SO₂N(R^{bb})₂, —SO₂R^{aa}, and —SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{x1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di- aliphaticamino, mono-or di-heteroaliphaticamino, mono- or di-alkylamino, mono- or di-heteroalkylamino, mono- or di-arylamino, or mono- or di-heteroarylamino; or two R^{x1} groups taken together form a 5- to 6-membered heterocyclic ring. Exemplary acyl groups include aldehydes (-CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers to a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C)═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

The term “oxo” refers to the group ═O, and the term “thiooxo” refers to the group ═S. Nitrogen atoms can be substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl, or two RCC groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, and R^{dd} are as defined above.

In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each nitrogen atom substituent is independently substituted (e.g., substituted with one or more halogen) or unsubstituted C₁-6 alkyl or a nitrogen protecting group.

In certain embodiments, each oxygen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or an oxygen protecting group. In certain embodiments, each oxygen atom substituents is independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each oxygen atom substituent is independently substituted (e.g., substituted with one or more halogen) or unsubstituted C₁₋₆ alkyl or an oxygen protecting group.

In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group.

Nitrogen, oxygen, and sulfur protecting groups are well known in the art and include those described in detail in Protecting Groups in Organic Synthesis, T. W. Greene and P. G. M. Wuts, 3rd edition, John Wiley & Sons, 1999, incorporated herein by reference.

In certain embodiments, the molecular weight of a substituent is lower than 250, lower than 200, lower than 150, lower than 100, or lower than 50 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, iodine, oxygen, sulfur, and/or nitrogen atoms. In certain embodiments, a substituent consists of carbon, hydrogen, fluorine, chlorine, bromine, and/or iodine atoms. In certain embodiments, a substituent consists of carbon, hydrogen, fluorine, and/or chlorine atoms. In certain embodiments, a substituent comprises 0, 1, 2, or 3 hydrogen bond donors. In certain embodiments, a substituent comprises 0, 1, 2, or 3 hydrogen bond acceptors.

The following definitions are more general terms used throughout the present application. As used herein, the term “salt” refers to any and all salts and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of this invention include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2—hydroxy—ethanesulfonate, lactobionate, lactate, laurate, lauryl sulfate, malate, maleate, malonate, methanesulfonate, 2—naphthalenesulfonate, nicotinate, nitrate, oleate, oxalate, palmitate, pamoate, pectinate, persulfate, 3—phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N±(C 1_4 alkyl)4 salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

A “peptide,” “polypeptide,” or “protein” comprises a polymer of amino acid residues linked together by peptide bonds. The terms refer to proteins, polypeptides, and peptides of any size, structure, or function. Typically, a protein will be at least three amino acids long. A protein may refer to an individual protein or a collection of proteins. Inventive proteins preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A peptide, polypeptide, or protein may also be a single molecule or may be a multi-molecular complex, may be a fragment of a naturally occurring protein or peptide, and may be naturally occurring, recombinant, synthetic, or any combination of these.

An “aminopeptidase enzyme” is a protein that catalyze the cleavage of amino acids from the amino terminus (N-terminus) of proteins or peptides (exopeptidases). Aminopeptidases are classified on the basis of their dependence on metal ions (usually Zn2+ or Mn2+) and substrate specificity. Most aminopeptidase enzymes remove one amino acid at a time, but certain aminopeptidases cleave two or three residues at a time; these are known as dipeptidyl and tripeptidyl aminopeptidases, respectively. See, e.g., Taylor A., Aminopeptidases: structure and function. FASEB Journal 7 (2): 290-8.

A “binder” is a molecule that specifically and reversibly interacts with the N-terminus of a protein or peptide. A binder may itself be a protein or peptide.

The term “polyethylenegycol” or “PEG” refers to a polyether compound by made by polymerizing ethylene glycol. PEG may be conjugated to a molecule (e.g., a peptide) in order to confer various advantageous properties such as solubility, surface active properties, and metabolic susceptibility. In certain particular embodiments, the PEG is less than 1000 g/mol. In certain particular embodiments, the PEG is between 1000-3000 g/mol. In certain particular embodiments, the PEG is between 2000-4000 g/mol. In certain particular embodiments, the PEG is between 3000-5000 g/mol. In certain particular embodiments, the PEG is between 4000-6000 g/mol. In certain particular embodiments, the PEG is between 5000-7000 g/mol. In certain particular embodiments, the PEG is between 6000-8000 g/mol. In certain particular embodiments, the PEG is between 7000-9000 g/mol. In certain particular embodiments, the PEG is between 8000-10000 g/mol. In certain particular embodiments, the PEG is greater than 10,000 g/mol. As used herein, the term “digestion” refers to enzymatic digestion. See, e.g., Riviere, L. R. and Tempst, P. (1995), Enzymatic Digestion of Proteins in Solution. Current Protocols in Protein Science, 00: 11.1.1-11.1.19.

### EMBODIMENTS

### Embodiment 1

A peptide comprising one or more instances of Formula (I):

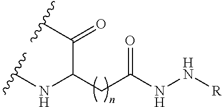

or a salt thereof, wherein:
each R is independently aryl, heteroaryl, or —C(O)Rₐ;
each Rₐ is independently branched or unbranched, cyclic or acyclic alkyl, branched or unbranched, cyclic or acyclic heteroalkyl, aryl, or heteroaryl; and
each n is independently 1 or 2.

### Embodiment 2

The peptide of Embodiment 1, wherein n is 1.

### Embodiment 3

The peptide of Embodiment 1, wherein n is 2.

### Embodiment 4

The peptide of any one of Embodiments 1-3, wherein R is aryl.

### Embodiment 5

The peptide of Embodiment 4, wherein R is phenyl.

### Embodiment 6

The peptide of any one of Embodiments 1-5, wherein Formula (I) has the structure:

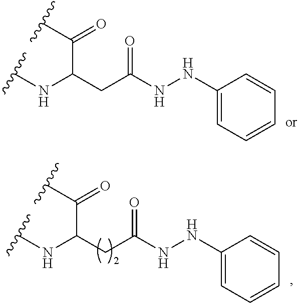

or a salt thereof.

### Embodiment 7

The peptide of any one of Embodiments 1-3, wherein R is heteroaryl.

### Embodiment 8

The peptide of any one of Embodiments 1-3, wherein R is —C(O)Rₐ.

### Embodiment 9

The peptide of any one of Embodiments 1-3, wherein R has the structure:

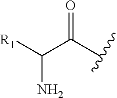

or a salt thereof, wherein R₁ is cyclic or acyclic alkyl, cyclic or acyclic heteroalkyl, aryl, or heteroaryl.

### Embodiment 10

The peptide of any one of Embodiments 1-9, wherein R comprises polyethyleneglycol (PEG).

### Embodiment 11

The peptide of Embodiment 9, wherein R₁ comprises PEG.

### Embodiment 12

The peptide of Embodiment 9, wherein R₁ is a natural amino acid side chain (e.g., a sidechain of glycine, alanine, valine, leucine, isoleucine, methione, phenylalanine, tryptophan, serine, threonine, glutamine, tyrosine, cysteine, lysine, arginine, histidine, aspartic acid, or glutamic acid).

### Embodiment 13

The peptide of Embodiment 12, wherein R₁ is isobutyl.

### Embodiment 14

The peptide of Embodiment 13, wherein Formula (I) has the structure of:

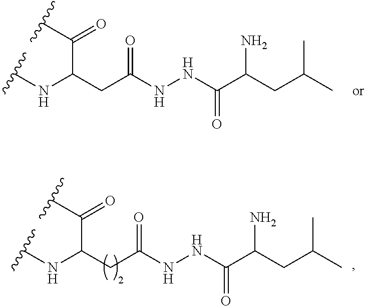

or a salt thereof.

### Embodiment 15

The peptide of any one of Embodiments 1-14, having a molecular weight greater than about 60 Da.

### Embodiment 16

The peptide of any one of Embodiments 1-15, wherein Formula (I) has the structure of Formula (II):

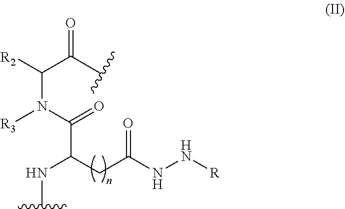

or a salt thereof, wherein:
each R₂ is independently branched or unbranched, cyclic or acyclic alkyl, branched or unbranched, cyclic or acyclic heteroalkyl, aryl, or heteroaryl;
each R₃ is -H, or is combined with R₂ to form a 5-membered heterocyclic ring; and
each n is independently one or two.

### Embodiment 17

A method for cleaving a peptide bond, comprising contacting a first peptide according to any one of Embodiments 1-16 with an aminopeptidase enzyme to obtain a second peptide comprising one or more instances of Formula (III):

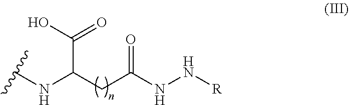

or a salt thereof.

### Embodiment 18

The method of Embodiment 17, wherein each peptide further comprises a moiety of Formula (IV):

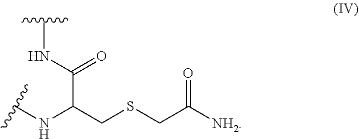

### Embodiment 19

The method of any one of Embodiments 17-18, wherein each peptide is conjugated to DNA.

### Embodiment 20

The method of any one of Embodiments 17-19, wherein the aminopeptidase enzyme is hTET, Vpr, or pfuTET.

### Embodiment 21

The method of any one of Embodiments 17-20, wherein the method further comprises contacting the first peptide with the aminopeptidase enzyme for 1-120 minutes.

### Embodiment 22

The method of any one of Embodiments 17-21, wherein the percent yield of the second peptide is in the range of about 10-100%, about 20-90%, about 40-90%, or about 60-80%.

### Embodiment 23

A method for modifying an aspartic acid residue or a glutamic acid residue in a peptide, the method comprising coupling a first peptide comprising a moiety of Formula (V):

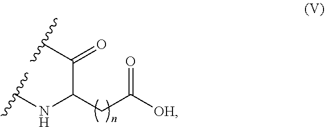

or a salt thereof;
wherein n is 1 or 2;
with a compound of Formula (VI):

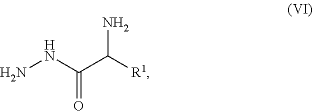

or a salt thereof;
wherein R₁ is cyclic or acyclic alkyl, cyclic or acyclic heteroalkyl, aryl, or heteroaryl;
to obtain a second peptide comprising a moiety of Formula (VII):

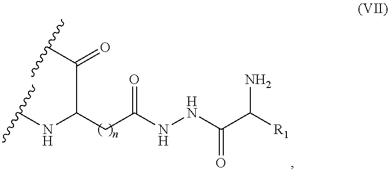

or a salt thereof.

### Embodiment 24

The method of Embodiment 23, wherein n is 1.

### Embodiment 25

The method of Embodiment 23, wherein n is 2.

### Embodiment 26

The method of any one of Embodiments 23-25, wherein R₁ comprises PEG.

### Embodiment 27

The peptide of any one of Embodiments 23-26, wherein R₁ is a natural amino acid side chain (e.g., a sidechain of glycine, alanine, valine, leucine, isoleucine, methione, phenylalanine, tryptophan, serine, threonine, glutamine, tyrosine, cysteine, lysine, arginine, histidine, aspartic acid, or glutamic acid).

### Embodiment 28

The method of any one of Embodiments 23-27, wherein R₁ is isobutyl.

### Embodiment 29

The method of Embodiment 28, wherein the moiety of Formula (VII) has the structure of:

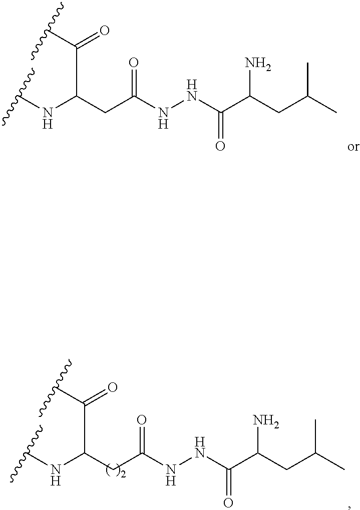 [SMILES: *NC(CC(=O)NNC(=O)C(N)CC(C)C)C(*)=O ; *NC(CC(=O)NNC(=O)C(N)CC(C)C)C(*)=O ; ","]

or a salt thereof.

### Embodiment 30

The method of any one of Embodiments 23-29, wherein the coupling comprises the use of a carbodiimide reagent.

### Embodiment 31

The method of Embodiment 30, wherein the carbodiimide reagent is immobilized on an insoluble solid support.

### Embodiment 32

The method of Embodiment 31, wherein the insoluble solid support is polystyrene.

### Embodiment 33

The method of any one of Embodiments 23-32, wherein the moiety of Formula (V) does not bind to a binder, and the moiety of Formula (VII) does bind to the binder.

### Embodiment 34

The method of Embodiment 33, wherein the first peptide and the second peptide further comprise an N-terminal amine, and the binder selectively binds to the moiety of Formula (VII) in favor of the N-terminal amine.

### Embodiment 35

The method of any one of Embodiments 33-34, wherein the binder is teClpS.

### Embodiment 36

A method of carbodiimide-mediated functionalization of a C-terminal carboxylate of a peptide, comprising reacting the peptide with an amine-containing molecule and a polystyrene (PS)-immobilized carbodiimide reagent, wherein a guanidinium by-product is formed through reaction of the amine-containing molecule and the PS-immobilized carbodiimide reagent, and wherein the guanidinium by-product is removed from the reaction mixture by filtration.

### Embodiment 37

The method of Embodiment 36, wherein the amine-containing molecule further comprises a click-chemistry handle, such as an azide, a tetrazine, a strained alkene, or an alkyne.

### Embodiment 38

The method of any one of Embodiments 36 and 37, wherein the amine-containing molecule is an oxime.

### Embodiment 39

The method of any one of Embodiments 36-38, wherein the amine-containing molecule has the structure:

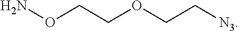

### Embodiment 40

The method of any one of Embodiments 36-39, wherein the PS-immobilized carbodiimide reagent has the structure:

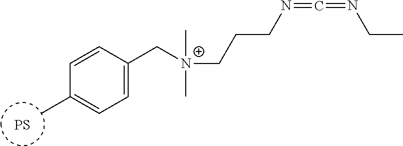

and optionally comprises a suitable counterion (e.g., lithium, sodium, potassium, or an ammonium).

### EXAMPLES

### Example 1

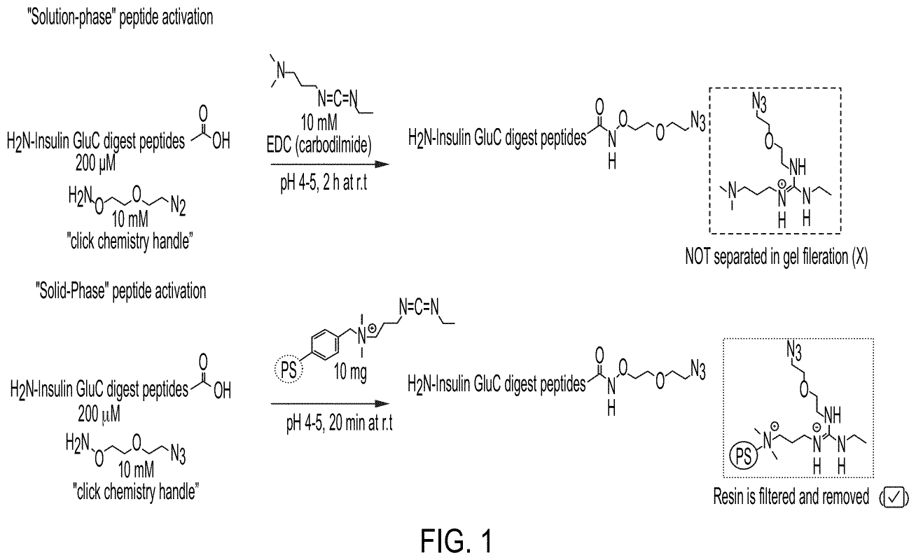

Prior the present disclosure, automation-compatible methods for peptide C-terminal immobilization have been limited to peptides bearing a C-terminal lysine residue. However, Carbodiimide activation of C-terminal carboxylate of peptides is a powerful technology that provides ability to modify the C-terminal carboxylates that are found in each peptide fragment generated using enzymatic digest. One challenge with automating carbodiimide coupling is the formation of a high-molecular weight adduct (byproduct). With reference to FIG. 1, disclosed herein is a solution to this challenge which involves immobilizing the carbodiimide reagent on a polystyrene (PS) resin that enables the filtration of the undesirable adduct. An additional advantage of PS-carbodiimide peptide activation is that all unreacted peptides remain covalently bound to the resin and are thereby easily removed from the reaction solution by filtration.

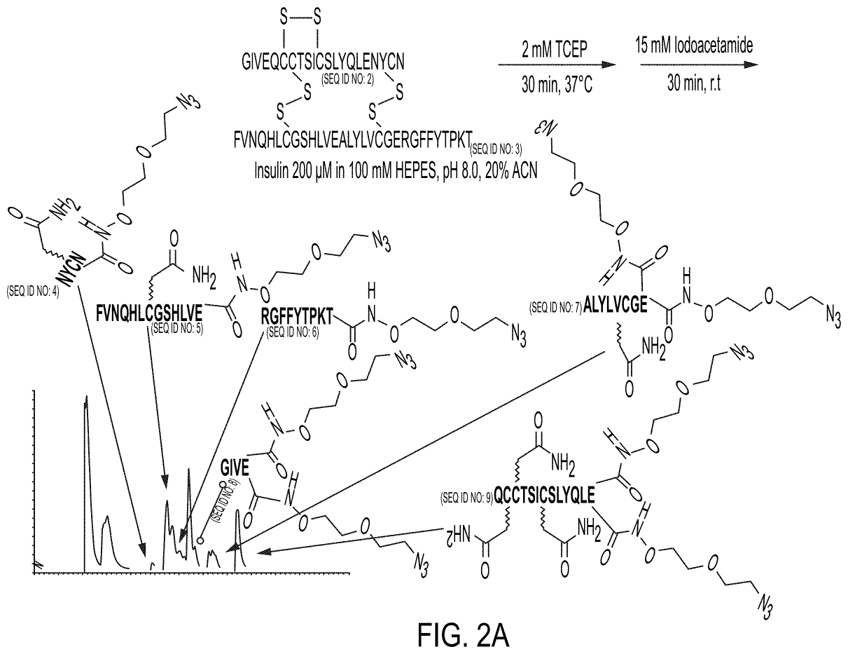

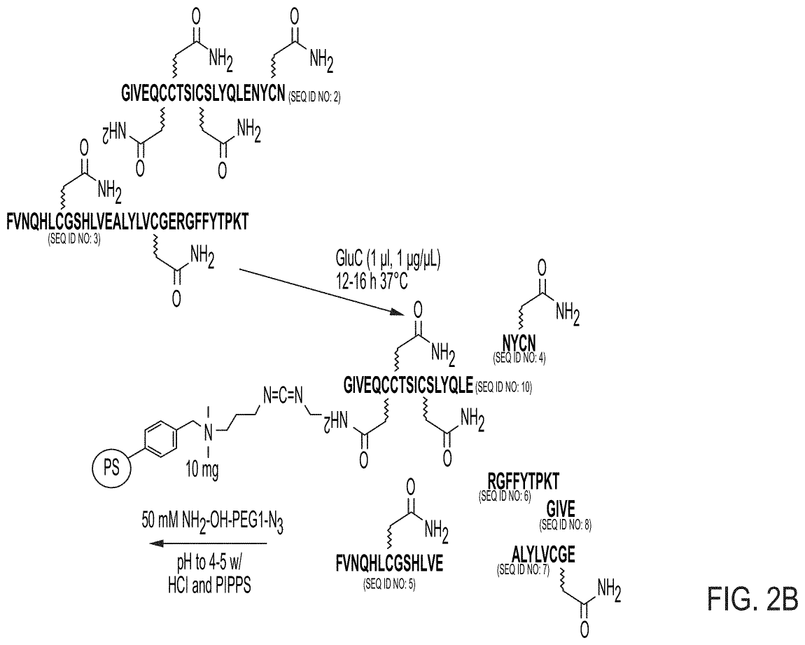

Carbodiimide C-terminal immobilization strategies currently rely on non-specific GluC digestion, which generates peptides with C-terminal Glutamate and Aspartate residues. Given the nature of such peptides, both carboxylic acids can be derivatized with a “click chemistry handle.” See, e.g., FIG. 1. The inventors observed bis-labeling when a one-pot GluC digestion of recombinant human insulin was prepared. See FIG. 2. With bis-labeling, either bis- or mono-labeled peptide will reach the aperture and will behave in a similar fashion. The peptide library presented in FIG. 2 represents the activation of all six different peptides that result from a GluC digest of recombinant human insulin.

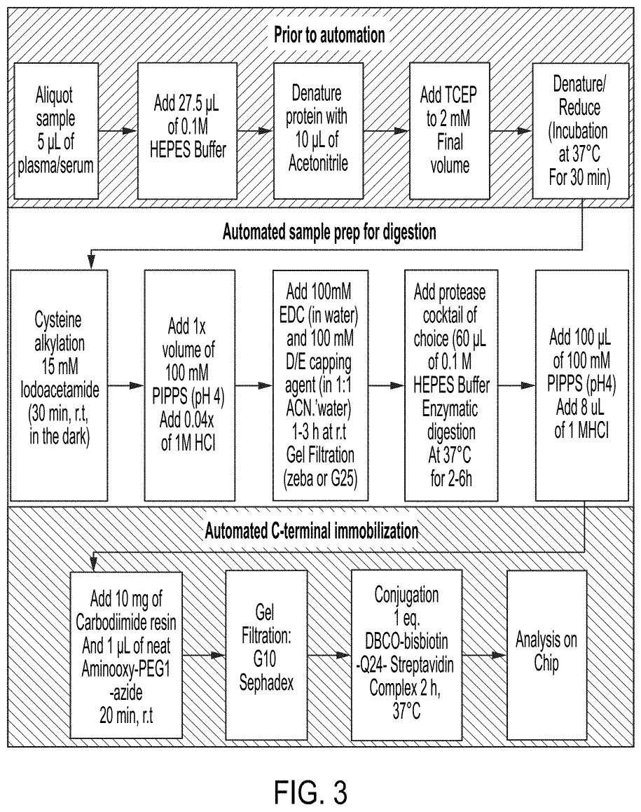

This C-terminal immobilization protocol can be used for any protein of interest when used in conjunction with the enzymatic (e.g., GluC) digestion. The flowchart of FIG. 3 represents the automation compatible protocol used to obtain libraries bearing C-terminally activated Asp/Glu peptide.

### Example 2

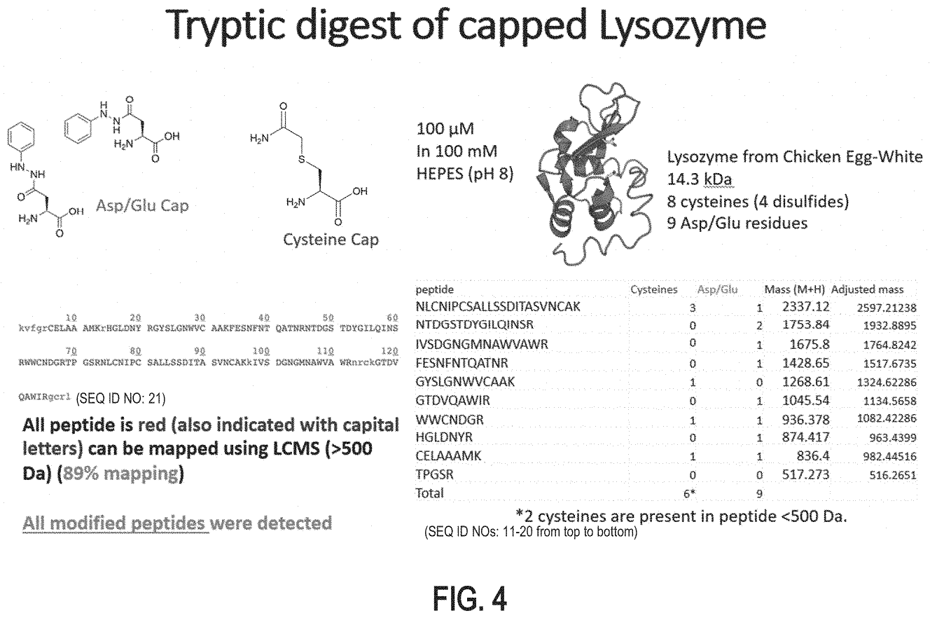

FIG. 4 shows aspartic acid (Asp) and glutamic acid (Glu) with a phenylhydrazine cap.

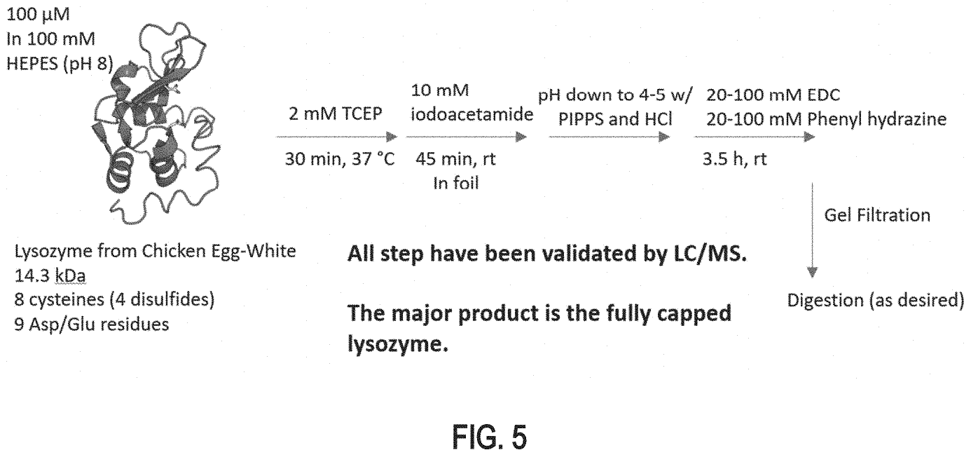

Also shown are peptides wherein the Asp and Glu residues are capped with phenylhydrazine and/or cysteine residues are capped with the cysteine cap shown. Mass (M+H) indicates the uncapped molecular mass of the peptide and Adjusted mass indicates the mass of the peptide with the indicated number of Asp/Glu caps and/or cysteine caps. Molecular mass was determined using an Ultimate3000LC-ExactivePlus Orbitrap High Resolution Mass Spectrometer (HRMS). An exemplary capping procedure is shown in FIG. 5.

1. A peptide comprising one or more instances of Formula (I): 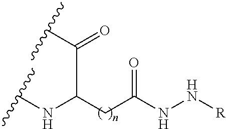
or a salt thereof, wherein:
each R is independently aryl, heteroaryl, or —C(O)Rₐ;
each Rₐ is independently branched or unbranched, cyclic or acyclic alkyl, branched or unbranched, cyclic or acyclic heteroalkyl, aryl, or heteroaryl; and
each n is independently 1 or 2.

2-3. (canceled)

4. The peptide of claim 1, wherein R is aryl.

5. (canceled)

6. The peptide of claim 1, wherein Formula (I) has the structure: 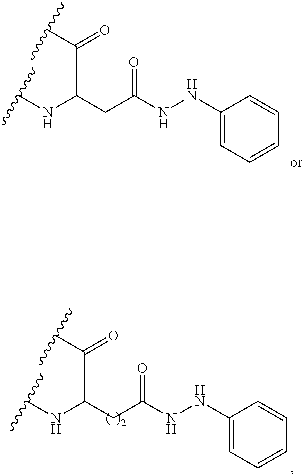 [SMILES: *NC(CC(=O)NNc1ccccc1)C(*)=O ; *NC(CC(=O)NNc1ccccc1)C(*)=O ; ","]
or a salt thereof.

7-8. (canceled)

9. The peptide of claim 1, wherein R has the structure: 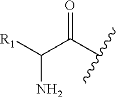
or a salt thereof, wherein R₁ is cyclic or acyclic alkyl, cyclic or acyclic heteroalkyl, aryl, or heteroaryl.

10-11. (canceled)

12. The peptide of claim 9, wherein R₁ is a natural amino acid side chain.

13. (canceled)

14. The peptide of claim 12, wherein Formula (I) has the structure of: 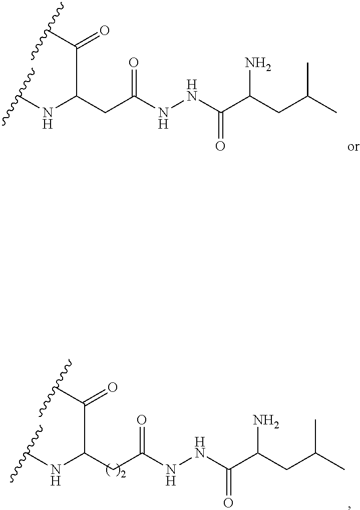 [SMILES: *NC(CC(=O)NNC(=O)C(N)CC(C)C)C(*)=O ; *NC(CC(=O)NNC(=O)C(N)CC(C)C)C(*)=O ; ","]
or a salt thereof.

15. (canceled)

16. The peptide of claim 1, wherein Formula (I) has the structure of Formula (II): 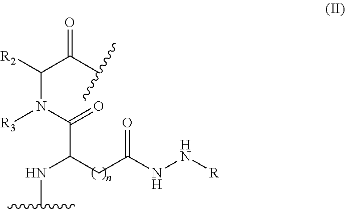
or a salt thereof, wherein:
each R₂ is independently branched or unbranched, cyclic or acyclic alkyl, branched or unbranched, cyclic or acyclic heteroalkyl, aryl, or heteroaryl;
each R₃ is -H, or is combined with R₂ to form a 5-membered heterocyclic ring; and
each n is independently one or two.

17. A method for cleaving a peptide bond, comprising contacting a first peptide according to claim 1 with an aminopeptidase enzyme to obtain a second peptide comprising one or more instances of Formula (III): 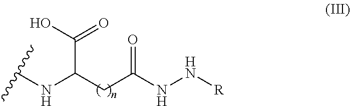
or a salt thereof.

18. (canceled)

19. The method of claim 17, wherein each peptide is conjugated to DNA.

20-22. (canceled)

23. A method for modifying an aspartic acid residue or a glutamic acid residue in a peptide, the method comprising coupling a first peptide comprising a moiety of Formula (V): 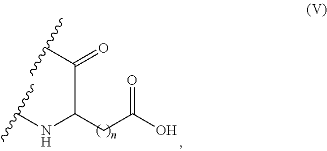 [SMILES: *NC(CC(=O)O)C(*)=O]
or a salt thereof;
wherein n is 1 or 2;
with a compound of Formula (VI): 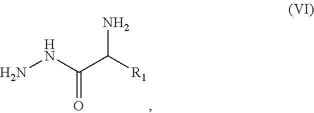
or a salt thereof;
wherein R₁ is cyclic or acyclic alkyl, cyclic or acyclic heteroalkyl, aryl, or heteroaryl;
to obtain a second peptide comprising a moiety of Formula (VII): 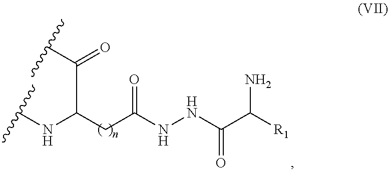
or a salt thereof.

24-26. (canceled)

27. The peptide of claim 23, wherein R₁ is a natural amino acid side chain.

28. (canceled)

29. The method of claim 27, wherein the moiety of Formula (VII) has the structure of: 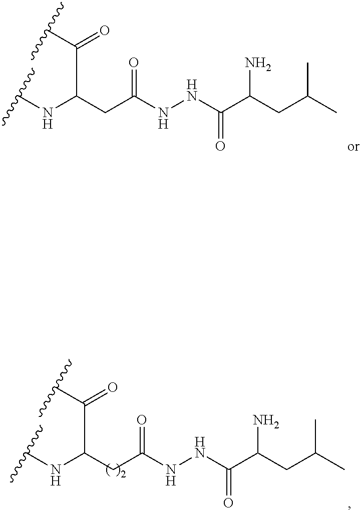 [SMILES: *NC(CC(=O)NNC(=O)C(N)CC(C)C)C(*)=O ; *NC(CC(=O)NNC(=O)C(N)CC(C)C)C(*)=O ; ","]
or a salt thereof.

30. The method of claim 23, wherein the coupling comprises the use of a carbodiimide reagent.

31. The method of claim 30, wherein the carbodiimide reagent is immobilized on an insoluble solid support.

32. (canceled)

33. The method of claim 23, wherein the moiety of Formula (V) does not bind to a binder, and the moiety of Formula (VII) does bind to the binder.

34. The method of claim 33, wherein the first peptide and the second peptide further comprise an N-terminal amine, and the binder selectively binds to the moiety of Formula (VII) in favor of the N-terminal amine.

35. The method of claim 33, wherein the binder is teClpS.

36. A method of carbodiimide-mediated functionalization of a C-terminal carboxylate of a peptide, comprising reacting the peptide with an amine-containing molecule and a polystyrene (PS)-immobilized carbodiimide reagent, wherein a guanidinium by-product is formed through reaction of the amine-containing molecule and the PS-immobilized carbodiimide reagent, and wherein the guanidinium by-product is removed from the reaction mixture by filtration.

37. The method of claim 36, wherein the amine-containing molecule further comprises a click-chemistry handle, such as an azide, a tetrazine, a strained alkene, or an alkyne.

38. (canceled)

39. The method of claim 36, wherein the amine-containing molecule has the structure:

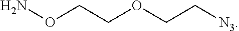

40. (canceled)
